(19) United States
(12) Patent Application Publication
(10) Pub. No.: US 2004/0034374 A1
(43) Pub. Date: Feb. 19, 2004

(54) Implant

(75) Inventors: Steffen Zatzsch, Freystadt (DE); Christoph Schopf, Mohrendorf (DE)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(73) Assignee: Tutogen Medical GmbH, Neunkirchen am Brand (DE)

(21) Appl. No.: 10/417,524

(22) Filed: Apr. 17, 2003

(30) Foreign Application Priority Data
Aug. 16, 2002 (DE) 10237531.3

(51) Int. Cl. A61B 17/08

(52) U.S. Cl. 606/151

(57) ABSTRACT

A hernia implant consists of a collagen membrane which is gained from biological starting material and is provided with a plurality of drainage openings.

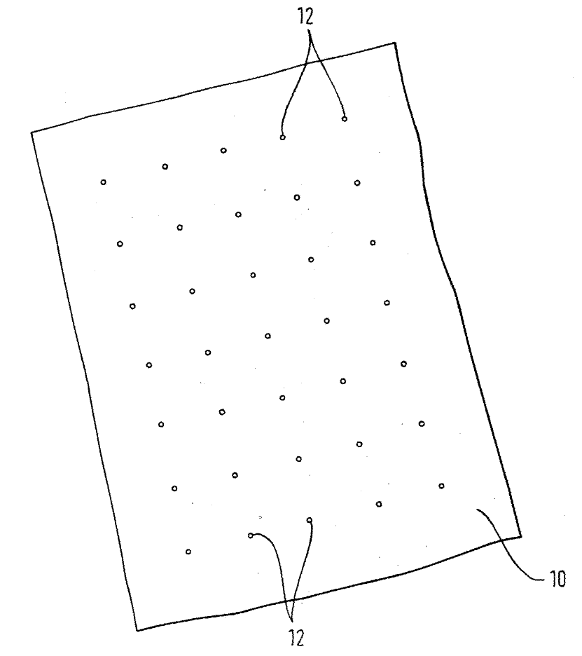

The present invention relates to a hernia implant for use in the field of hernia surgery.

A hernia is the protuberance of the peritoneum through a gap in the abdominal wall, also called a rupture. Depending on the localization of the hernia orifice, one distinguishes a variety of hernias such as inguinal hernias, umbilical hernias, femoral hernias and incisional hernias.

In addition to general connective tissue weakness, above all the passage points of the umbilical cord, of the spermatic cord and of the large blood vessels promote a hernia formation. With incisional hernias, a cause is the very long healing of the abdominal incision, as well as the mechanical fatigue of the devitalized scar.

According to the present state of medical knowledge, almost exclusively synthetic materials are used to cover the hernia gap or to strengthen the abdominal wall in hernia operations. Non absorbable meshes and membranes are substantially used here. Absorbable meshes are only used rarely due to the fast loss of tearing force.

Known non absorbable meshes consist of polypropylene, polyethylene terephthalate or polytetrafluorethylene. Due to tissue reactions of the body, such plastic meshes are encapsulated like connective tissue. The thickness of the capsule here is directly proportional to the amount of foreign material introduced. The capsules created shrink over time which is why the meshes have to overlap the rims of the hernia gap by at least approximately 5 to 8 cm. Due to the extended preparation required here, the risk of hematomas or seromas is pronounced. In addition, migration of the meshes through the abdominal wall can occur due to different pressure distributions.

Generally non absorbable meshes are prone to implant infections, which applies in particular to closely knit meshes and to fine pored membranes. An explantation is often necessary due to mesh infections.

Due to the aforesaid problems, the reoperation rate lies at approximately 5 to 20% on the use of absorbable and non absorbable meshes.

It is the object of the present invention to provide a hernia implant which eliminates the aforesaid problems.

This object is satisfied by the features of claim 1 and in particular by a hernia implant which consists of a collagen membrane which is gained from biological starting material and which is provided with a plurality of drainage openings spaced apart from one another.

The hernia implant in accordance with the invention is gained, for example, from bovine pericardium, human pericardium or from fascia lata and conserved as well as sterilized. The drainage openings are provided for the protection of the implant itself, unlike conventional hernia meshes in which the mesh shape serves for the reduction of the amount of foreign material introduced. The inventors have namely recognized that post-operatively arising wound exudate, so-called seroma, contains an increased number of macrophages and these effect an unwanted breakdown of the collagen structure. To prevent the implant being broken down faster than it is converted into the body's own connective tissue, in accordance with the invention a draining of the seroma fluid from the intermediate space between the musculature and the implant is ensured in that the drainage openings spaced apart from one another are provided.

The hernia implant in accordance with the invention can either be sutured in abutment or be placed on with an only small overlap of approximately 1 cm, since it does not shrink. This allows—in comparison with conventional implants—a use of smaller implants, whereby less foreign material is implanted into the body and, in addition, substantial costs are saved. The implant is further very largely infection resistant and it does not migrate within the body, since it has the same biomechanics as the abdominal wall itself. The implant can be implanted in direct contact with the intestines without causing inflammation or adhesion. The conversion of the implant into connective tissue takes place within the framework of the wound healing, whereby only a low risk of recurrence is given.

The implant, which consists exclusively of a collagen membrane, is very soft and supple. There are no sharp or hard edges which could trigger irritations. A further advantage is that the implant is converted into vital connective tissue of the body in the course of the continuously proceeding conversion process.

The target indications of the hernia implant in accordance with the invention are various. For instance, an erosion of the spermatic cord can be avoided in male patients and a hindrance to natural stretching during pregnancy can be avoided in female patients.

A further field of application lies in the replacement of infected meshes. Since, when plastic meshes are used, the risk of an infection due to the specific properties of these meshes is high, complications can partly occur even after years, with these meshes having to be removed and replaced by other materials. Since a repeated use of a plastic mesh in the infected area would be contra-indicated, the hernia implant in accordance with the invention can be used in an advantageous manner here.

A further advantageous application area is the repair of incisional hernias which can in particular occur after a midline incision of the abdominal wall and can be of large extent.

Advantageous embodiments of the invention are described in the description, in the drawing as well as in the dependent claims.

In accordance with a first advantageous embodiment, the drainage openings of the implant can be uniformly spaced apart from one another and in particular be distributed in the manner of a net-like grid. It is hereby ensured that the seroma can flow off uniformly and over the whole area, whereby the collagen membrane is protected over the whole area.

The drainage openings can have a mutual spacing of approximately 5 to 15 mm and in particular be made as round perforations. It has been found to be advantageous here if the drainage openings have a diameter, or a flow width, of approximately 0.5 to 1.5 mm.

A particularly advantageous embodiment results when the drainage openings are substantially distributed along a square grid over the whole collagen membrane, with a marginal zone being able to be provided which is not provided with drainage openings.

It is furthermore advantageous for the healing in process if the hernia implant consists only of the collagen membrane, which is preferably made in one layer.

An embodiment of the invention will be described in the following purely by way of example with reference to the enclosed drawing.

The only FIGURE shows a perspective view of a hernia implant.

The hernia implant shown in the FIGURE consists of a single layer collagen membrane 10 which is gained from biological starting material, for example from bovine pericardium. The collagen membrane 10 is rectangular and has dimensions of approximately 4×5 cm. Further conceivable sizes are 6×8 cm, 8×12 cm and 12×16 cm. Other sizes and shapes, e.g. round shapes or shapes provided with slots, can likewise be advantageous. The collagen membrane 10 shown in the FIGURE has a plurality of drainage openings 12 which are arranged along a square grid and which have a mutual spacing of approximately 10 mm. Each drainage opening 12 is made as a round perforation with a diameter of approximately 1 mm. A peripheral marginal zone with a width of approximately 10 mm is made free of perforations. 35 drainage openings are provided in the embodiment shown.

1. A hernia implant consisting of a collagen membrane which is gained from a biological starting material and is provided with a plurality of drainage openings spaced apart from one another.

2. A hernia implant in accordance with claim 1, characterized in that the drainage openings are uniformly spaced apart from one another.

3. A hernia implant in accordance with claim 1, characterized in that the drainage openings are distributed in the manner of a net-like grid.

4. A hernia implant in accordance with claim 1, characterized in that the drainage openings have a mutual spacing of approximately 5 to 15 mm.

5. A hernia implant in accordance with claim 1, characterized in that the drainage openings are round perforations.

6. A hernia implant in accordance with claim 1, characterized in that the drainage openings have a flow width of approximately 0.5 to 1.5 mm.

7. A hernia implant in accordance with claim 1, characterized in that the ratio of the mutual spacing to the flow width of the drainage openings is approximately ten to one.

8. A hernia implant in accordance with claim 1, characterized in that the drainage openings are substantially distributed over the whole collagen membrane.

9. A hernia implant in accordance with claim 1, characterized in that the collagen membrane is gained from bovine or human pericardium or from fascia lata.

10. A hernia implant in accordance with claim 1, characterized in that it only consists of collagen membrane which is preferably made in one layer.

11. A hernia implant in accordance with claim 1, characterized in that it consists of native, non cross-linked, absorbable collagen.

12. A hernia implant in accordance with claim 1, characterized in that it is subjected to a validated process for disantigenization and sterilization.
